(19) United States
(12) Patent Application Publication
(10) Pub. No.: US 2012/0134950 A1
(43) Pub. Date: May 31, 2012

(54) Adhesion-Promoting thin Film and Moisture Resistant Skin Barrier Compositions

(76) Inventors: Steven J. Babb, Seminole, FL (US); Dennis M. Kay, Tampa, FL (US)

(21) Appl. No.: 13/284,053

(22) Filed: Oct. 28, 2011

Related U.S. Application Data
(60) Provisional application No. 61/407,533, filed on Oct. 28, 2010.

(51) Int. Cl. A61K 31/74; A61P 31/02; A61K 31/14; A61K 47/10; A61K 31/765

(52) U.S. Cl. 424/78.02; 514/772; 514/643

(57) ABSTRACT

Adhesion-promoting, thin film, moisture resistant skin barrier formulations are composed of a fugitive solvent mixture consisting of t-butanol and an alkane fraction composed of at least one alkane, where the t-butanol is in the range of 40-70% by weight of the solvent mixture, and a film-forming solute dissolved in the fugitive solvent mixture. The formulations may also include one or more optional ingredients such as antimicrobial and/or bioactive agents. When applied to the skin, the formulations improve the adhesion of various classes of medical adhesives used to attach a variety of medical devices to the skin and create a thin, flexible and elastic continuous barrier film on the skin to protect against body fluids and incontinence.

### CROSS-REFERENCE TO RELATED PATENT APPLICATION

This application claims priority from U.S. provisional patent application Ser. No. 61/407,533 filed Oct. 28, 2010, the entire disclosure of which is incorporated herein by reference.

### BACKGROUND OF THE INVENTION

1. Field of the Invention

The present invention relates to chemical formulations/compositions of a no-sting, quick-drying, film-forming solution which is useful in promoting the holding strength of medical adhesives and providing a flexible and elastic barrier on the skin to protect against incontinence and body fluids.

2. Brief Discussion of the Related Art

The prior art for skin barrier film formulations is based on various solvent systems each having disadvantages compared to the formulations/compositions of the present invention. Examples of early prior art, as represented by U.S. Pat. No. 4,942,029 to Scheps, required harsh, stinging solvents for the barrier polymer such as isopropyl alcohol, ethyl alcohol, ethyl acetate, acetone, and various other polar solvents. The prior art includes many variations of stinging, polar solvent and barrier film polymer combinations.

To alleviate stinging and irritation, subsequent prior art contains examples of solvent chemistry which are either water-based or siloxane-based. The water-based formulations, as a class, have the disadvantages of negative impact on adhesive strength caused by hydrated skin, not having solvating power to remove oils and waxes from the skin, and evaporating slowly. The water-based formulations also leave a barrier film which is at least partially soluble in water and thereby rendered partially ineffective by contact with bodily fluids and incontinence.

The siloxane formulations exemplified by the commercially available products known as 3M Cavilon™ and Silesse™ are no-sting. The siloxane formulations, as exemplified by U.S. Pat. No. 4,987,893 to Salamone et al, U.S. Pat. No. 7,318,937 to Jonn et al and U.S. Pat. No. 6,383,582 to Dunshee et al, are disadvantageous because they are not particularly effective in promoting adhesion on skin. Siloxane formulations have the further disadvantage of reducing hydrocolloid adhesion as compared with the formulations/compositions of the present invention. The prior art in the siloxane formulation class also typically includes emollients which cause additional skin moisturization with attendant reduction in hydrocolloid adhesion. Cyclopentasiloxane and other cyclic siloxanes are typical prior art emollients and are deleterious to hydrocolloid adhesion.

U.S. Pat. No. 3,928,556 to Sweger describes use of mixtures of t-butanol and branched alkanes including iso-octane as a non-stinging solvent for use in wound dressings that are relatively thick. Sweger does not contemplate use of these compounds in adhesion-promoting barrier films and does not disclose useful formulations containing less than 50% t-butanol. In addition, Sweger is not concerned with adhesion promotion of medical adhesives. Sweger describes formulations containing up to 30% polymer (plastic) and does not consider the advantages of a very low concentration of dissolved polymer to form a low viscosity solution, thereby enhancing a more cohesive bond to the irregularities of skin which is crack/crumble resistant, and forms an ultra thin “tie layer” to enhance the bonding capabilities of medical adhesives and the occlusive seal of medical devices.

### SUMMARY OF THE INVENTION

The present invention concerns adhesive-promoting, thin film, moisture resistant chemical formulations for application to the skin without stinging and irritation. The formulations can be applied to the skin in various ways. The formulations promote the adhesive strength or bonding of medical adhesives, and particularly hydrocolloid adhesives, used to attach a variety of medical devices to the skin. The formulations evaporate especially quick on the skin, enabling attachment of the adhesive medical device immediately after application of the formulation to the skin. The formulations create a thin, flexible and elastic continuous barrier film on the skin to protect against body fluids and incontinence. The barrier film created with the formulations is crack-resistant and crumble-resistant. The formulations have a low viscosity so that a solution of the formulation is capable of penetrating and filling the fine textural features of skin.

The formulations are composed of a fugitive solvent mixture made up of t-butanol and an alkane fraction or component made up of at least one alkane, wherein the t-butanol is in the range of 40-70% by weight of the solvent mixture, and a film-forming solute dissolved in the fugitive solvent mixture. Preferably, the t-butanol is less than 50% by weight of the formulation. The formulations may further include one or more optional ingredients, such as antimicrobial and/or bioactive agents.

The at least one alkane used in the solvent mixture is preferably iso-octane, but other alkanes can be used as the alkane fraction in the solvent mixture individually or in combination. The alkane fraction of the solvent mixture is in the range of 30-60% by weight of the solvent mixture. The film-forming solute can be a film-forming copolymer, particularly the butyl ester of poly-ethyl-vinyl-ether/maleic acid known commercially as Gantrez® ES-425. However, other film-forming solutes are compatible with the formulations, and the film-forming solute may optionally include a suitable plasticizer. The optional ingredient may be benzalkonium chloride to serve as an antimicrobial agent, but various other antimicrobial and/or bioactive agents are compatible with the formulations. The amount of film-forming solute used in the formulations may be varied from a relatively low concentration to obtain an ultra-thin barrier film on skin to a relatively high concentration to obtain a barrier film providing increased moisture resistance. Specific examples of formulations are described herein.

Various objects, advantages and benefits of the present invention will become apparent from the following detailed description of the invention taken in conjunction with the accompanying drawings.

### BRIEF DESCRIPTION OF THE DRAWINGS

FIG. 1 is a bar graph of test results showing the adhesive strength on human skin obtained for a formulation of the present invention and several prior art products.

FIG. 2 illustrates the chemical structure of a film-forming copolymer used as the film-forming solute in a formulation of the present invention.

FIG. 3 is a bar graph illustrating the evaporation time on human skin for a formulation of the present invention and several prior art products.

FIG. 4 is a graph illustrating the solubility of constituents of the formulation for variable temperatures and percentages by weight of iso-octane.

FIG. 5 is a graph illustrating evaporation times for the iso-octane/t-butanol solvent mixture for variable percentages by weight of iso-octane.

### DETAILED DESCRIPTION OF THE INVENTION

The invention pertains to no-sting, quick-drying, film-forming chemical formulations or compositions for being applied to skin and which promote the holding strength of medical adhesives on the skin and provide a flexible and elastic barrier on the skin to protect against incontinence and body fluids.

The enhanced adhesion provides additional reliability for attachment of medical devices to skin. The solvent mixture in the formulation evaporates nearly instantly on skin to facilitate immediate application of an adhesive medical device to the skin. The formulation has solvent or dispersion capabilities for a spectrum of optional antimicrobial and/or biologically active agents or compounds, and the inclusion of such agents in the formulation is explained further below. The formulation is stable and compatible with sterilization processing. The barrier film-forming component of the formulation has low solubility to incontinence and body fluids.

Medical devices that may make use of the formulations include catheter securement, ostomy and urological devices, bandages, prosthetic, electrode and ultrasonic securement pads, wound dressings, and other devices which are adhesively affixed to skin. Appropriate methods for delivery of a solution of the formulations to skin include wipe (towelette, pad, sachet), spray bottle (pump or pressurized), ampoule, and swab.

Unlike the prior art discussed above, the formulations of the present invention are designed specifically to improve adhesion of medical devices and particularly hydrocolloid adhesion, and can vary the concentration of film-forming solute in a solvent mixture over a wide range, such as a low concentration for an ultra-thin adhesion-promoting film, a higher concentration to achieve optimal moisture resistance thereby extending the wear time of a medical device on skin, and yet other intermediate concentrations.

The formulations of the present invention provide the advantages of reducing excess skin hydration and removing waxes and oils from skin to optimize adhesive bonding, particularly as this applies to hydrocolloid adhesives. These advantages are achieved in the formulations by means of a fugitive solvent mixture consisting of a neutral polarity alkane fraction or component, e.g. iso-octane, and a low-polarity t-butanol fraction or component, wherein the t-butanol is at least 40% by weight of the solvent mixture. In an aspect of the present invention, the formulations are made from a fugitive solvent mixture containing t-butanol in the range of 40-70% by weight of the solvent mixture, with the balance being an alkane fraction made up of at least one alkane, e.g. iso-octane, in the range of 30-60% by weight of the solvent mixture. The t-butanol in the formulations is preferably less than 50% by weight of the formulation. The formulations also provide the advantages of formulations which evaporate nearly instantaneously on the skin and provide a chemically-inert and nearly neutral polarity solvent system which permits dissolution of a wide spectrum of optional antimicrobial and/or bioactive agents or compounds from neutral to highly polar.

The formulations of the present invention improve the adhesion of various classes of medical adhesives especially pressure-sensitive adhesives including hydrocolloid adhesives, various vinyl or acrylic-based adhesives such as polyvinyl-methacrylate (PVM), polyvinyl-alcohol (PVA), and silicone-based adhesives. However, the adhesion promotion obtained with the present invention is not limited to these classes of medical adhesives.

The formulations of the present invention are particularly effective in promoting the holding strength of hydrocolloid adhesives. The formulations provide a skin barrier film that is effective in preventing irritation and skin breakdown from urinary and fecal incontinence. The formulations can be designed for optimal moisture resistance to optimally maintain the barrier film in the presence of significant moisture, thereby extending the wear time of various medical devices including ostomy devices.

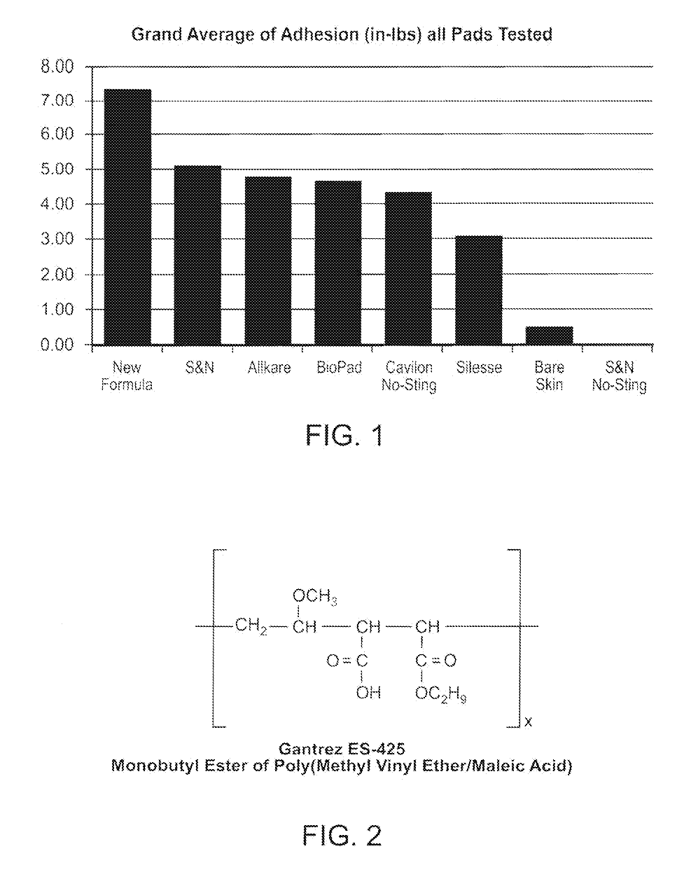

As one example of the hydrocolloid adhesion promotion realized with the present invention, the adhesive strength of hydrocolloid adhesive on human skin obtained with a formulation of the present invention has been compared in testing to several other products which are prior art, namely, Smith & Nephew (S&N™), Allkare®, BioPad®, Cavilon™ No-Sting, Silesse™, BareSkin (no barrier film), and S&N No-Sting™. The test results are shown in FIG. 1 and quantify that the adhesive strength obtained with the formulation of the present invention (New Formula) was at least 50% greater or more than each example of prior art product. In particular, the test results demonstrate an adhesive strength greater than 7 in.-lbs. for the formulation of the present invention.

Formulations of the present invention are composed of a film-forming solute dissolved into a fugitive (evaporating) solvent mixture consisting of t-butanol (also known as tert-butanol, tertiary butyl alcohol, 2-methyl-2-propanol, trimethylcarbinol) and an alkane fraction composed of at least one alkane, plus additional optional ingredients such as antimicrobial and/or bioactive agents. In one aspect of a formulation of the present invention, the formulation is composed of a film-forming copolymer dissolved into a fugitive solvent mixture of t-butanol and iso-octane (also known as isobutyltrimethylmethane, 2,2,4-trimethyl-pentane), plus additional optional ingredients such as antimicrobial and/or bioactive agents. The following Example 1 sets forth the composition for one aspect of a formulation of the present invention particularly advantageous for providing an ultra-thin, adhesion-promoting, barrier film on skin.

### Example 1

### Composition for Ultra-Thin Adhesion-Promoting Barrier Film

| | wt. % |
|---|---|
| A. Iso-octane | 55.0 |
| B. t-Butanol | 41.1 |
| C. Butyl Ester of PVM/MA Copolymer, 50% in ethanol | 3.4 |
| D. Benzalkonium Chloride (BZK), 50% Solution | 0.5 |
| | 100% |

Although iso-octane is the alkane used for the alkane fraction of the solvent mixture in the formulation of Example 1, other suitable alkanes may be substituted for the iso-octane used for the alkane fraction of the solvent mixture, such as hexane, heptane, nonane, and decane, individually or in combination. The alkane(s) employed may be in the form of various isomers (n-, iso-, etc.) and in various combinations. Iso-octane is preferred due to the favorable evaporation rates achieved on skin.

In the formulation of Example 1, the film-forming copolymer is butyl ester of poly-vinyl-methyl-ether/maleic acid, which is commercially available as Gantrez® ES-425 sold by ISP Technologies, 1361 Alps Rd., Wayne, N.J. 07470. The chemical structure of Gantrez® ES-425 is set forth in FIG. 2. The Gantrez® ethyl ester known as ES-225 may optionally be used.

The butyl ester of poly-vinyl-methyl-ether/maleic acid used in the formulation for the film-forming solute is capable of forming ultra-thin, continuous barrier films on skin that exhibit elasticity and flexibility similar to normal human skin, without addition of a plasticizer. The barrier film is inherently crack-resistant to the conformational changes of skin, and this property is achieved without added plasticizer. In contrast, prior art formulations result in cracking and caking-up, which reduces the occlusive seal and wear time of hydrocolloid adhesives.

The low viscosity of the formulations exemplified by Example 1 allows an ultra-thin film to be deposited on skin, which is more crack-resistant and crumble-resistant than prior products, thereby enhancing a more cohesive bond to the irregularities of skin and providing a more effective co-adhesive “tie layer” to enhance the bonding capabilities of medical adhesive devices and the occlusive seal of hydrocolloid medical devices. A low viscosity (less than 3 centipoise) solution of the formulation is capable of penetrating and filling the fine textural features of skin to leave a continuous and completely bonded thin film. The integrity of the crack-free and high strength bonding adhesion between the barrier film and hydrocolloid lamination is thereby enhanced.

Other film-forming solutes, including film-forming polymers, copolymers, terpolymers etc. are also compatible with the formulations including, for example, poly-vinyl-alcohol (PVA) and poly-vinyl-methyl-ether/maleic acid copolymer, acrylate terpolymers, poly-vinyl pyrrolidone (PVP), and various polymers and copolymers preferentially containing moieties that enhance skin adhesion, such as carboxyl, ester, acrylic, and 2- and 3-pyrrolidone. Plasticizing ingredients may be added to the film-forming solute to enhance flexibility of the film deposited on the skin. Suitable plasticizers include, but are not limited to, acetyl tributyl citrate and citric acid.

In the aspect of the formulation set forth in Example 1, the benzalkonium chloride (BZK) serves as an optional antimicrobial agent. Various other antimicrobial and/or biologically active agents or compounds are compatible with the formulation including, for example, triclosan (poly-chlorophenoxy-phenol), CHG (chlorhexidine gluconate), and sulfa (sulfonamide) class antibiotics.

The use of the mixture of an alkane fraction composed of at least one alkane, e.g. iso-octane, and t-butanol to form the fugitive solvent mixture, with the t-butanol being in the range of 40%-70% by weight of the fugitive solvent mixture, accounts for several features of the present invention. The t-butanol provides only a slight polarity to the otherwise neutral and chemically inert iso-octane or other alkane fraction of the solvent mixture. The neutral polarity provided by the iso-octane or other alkane fraction of the solvent mixture is suitable for dissolution and removal of waxes and oils on the skin, which is beneficial to hydrocolloid adhesion. The low-polarity t-butanol in the formulation is suitable for mild dehydration of the top epidermal layers, which also benefits hydrocolloid adhesion. The components of the solvent mixture are each, and in combination, sting-free to either intact or damaged skin, mucosal membranes, urethral openings, and lacrimal ducts, for example.

The chemically inert/very low polarity of the solvent mixture also results in compatibility with various antimicrobial and biologically active optional ingredients as previously described. The low polarity of the solvent mixture also results in a balanced formulation which beneficially dehydrates the top epidermal layers of the skin due to extraction of the polar water molecules by the t-butanol, and removes waxes and oils of nearly neutral polarity due to extraction by the neutral polarity iso-octane or other alkane fraction. Removal of both excess moisture and oils/waxes is highly beneficial to adhesive bonding, particularly with hydrocolloid adhesives.

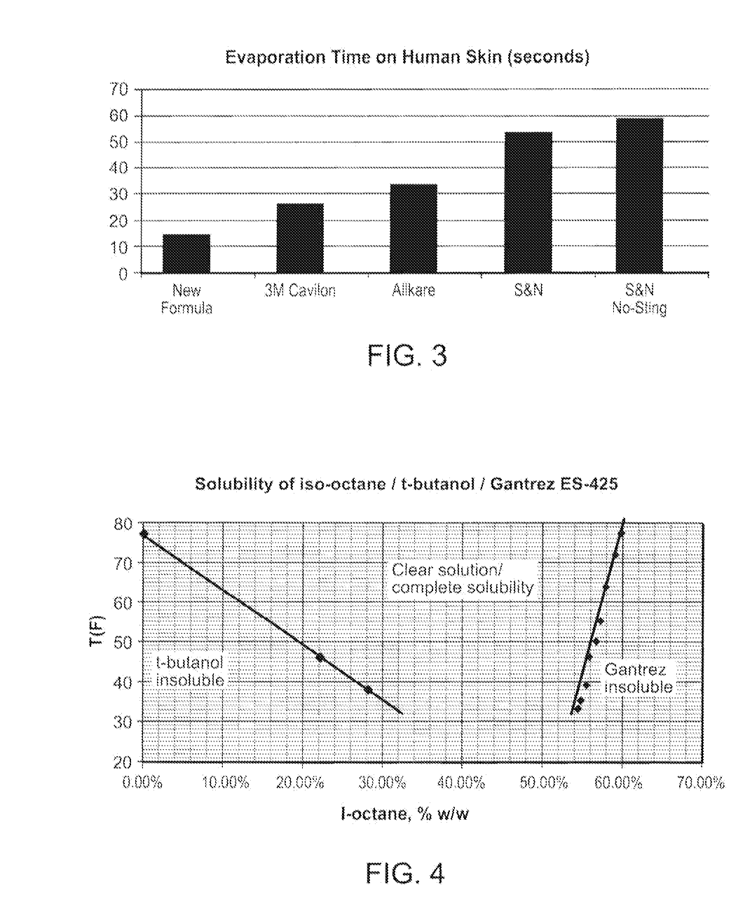

Once applied to the skin, the fugitive solvent mixture evaporates nearly instantly, which is an advantage because near instantaneous evaporation allows a medical device to be applied immediately after delivery of the formulation to the skin. FIG. 3 depicts the evaporation time on human skin for a formulation of the present invention (New Formula) and several prior products, namely, 3M Cavilon™, Allkare®, S&N and S&N No-Sting™. The data presented in FIG. 3 show that the formulation of the present invention has an evaporation time on skin of less than 10 seconds and, more specifically, less than about 8 seconds, whereas the prior art products take about 25 seconds and longer to evaporate on skin. Depending on the percentages of constituents used in the formulations of the invention, the evaporation time on skin may be longer than 10 seconds but no longer than 20 seconds.

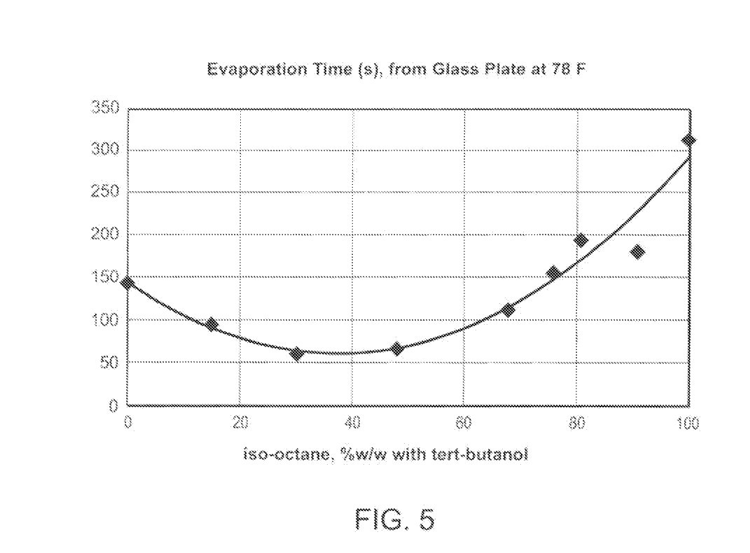

In variations of the preferred solvent mixture, the alkane fraction, e.g. iso-octane, may be varied ideally in the range of 30-60% by weight of the solvent mixture, with the balance of the solvent mixture being t-butanol in the range of 40-70% by weight of the solvent mixture. This provides for a stable solution while in storage down to 32° F. as shown by the graph of FIG. 4, and is in the optimal range for rapid evaporation of the solvent system as shown by the graph of FIG. 5. The optimal composition of an alkane fraction, e.g. iso-octane, and t-butanol in the solvent mixture in the described range results in a much faster evaporation rate than either component alone.

In another aspect of a formulation of the present invention, where the aim is to produce a film that is optimally resistant to moisture, the concentration of film-forming solute may be substantially increased as set forth below in Example 2. Such a moisture-resistant film is particularly advantageous for use with, but not limited to, ostomy appliances that may present bodily fluids to the skin adhesive interface and application of medical devices, including catheter securement devices, onto diaphoretic skin.

### Example 2

### Composition for Adhesion-Promoting Optimally Moisture Resistant Barrier Film

| | wt. % |
|---|---|
| A. Iso-octane | 44.0 |
| B. t-Butanol | 33.5 |
| C. Butyl Ester of PVM/MA Copolymer, 50% in ethanol | 22.0 |
| D. Benzalkonium Chloride (BZK), 50% Solution | 0.5 |
| | 100% |

The higher concentration of film-forming solute, i.e. butyl ester of PVM/MA copolymer in Example 2, provides a relatively thicker (compared to ultra-thin) sealing barrier film that accounts for optimal moisture resistance. The high concentration of film-forming solute, e.g. copolymer, achieved in the formulation that accounts for the optimally moisture resistant barrier film is surprisingly high for a nearly neutral polarity solvent.

Inasmuch as the present invention is subject to many variations, modifications and changes in detail, it is intended that all subject matter discussed above or shown in the accompanying drawings be interpreted as illustrative only and not be taken in a limiting sense.

What is claimed is:

1. An adhesion-promoting, thin film, moisture resistant skin barrier formulation comprising
a fugitive solvent mixture consisting of t-butanol and an alkane fraction composed of at least one alkane, wherein the t-butanol is at least 40% by weight of the fugitive solvent mixture; and
a film-forming solute dissolved in the fugitive solvent mixture.

2. The adhesion-promoting, thin film, moisture resistant skin barrier formulation recited in claim 1 wherein the t-butanol is in the range of 40-70% by weight of the fugitive solvent mixture.

3. The adhesion-promoting, thin film, moisture resistant skin barrier formulation recited in claim 2 wherein said alkane fraction consists of iso-octane.

4. The adhesion-promoting, thin film, moisture resistant skin barrier formulation recited in claim 2 wherein said alkane fraction is composed of one or more of iso-octane, hexane, heptane, nonane or decane, or isomers thereof.

5. The adhesion-promoting, thin film, moisture resistant skin barrier formulation recited in claim 2 wherein said film-forming solute is composed of one or more of a film-forming polymer, copolymer or terpolymer.

6. The adhesion-promoting, thin film, moisture resistant skin barrier formulation recited in claim 5 wherein said film-forming solute consists of butyl ester of poly-vinyl-methyl-ether/maleic acid copolymer.

7. The adhesion-promoting, thin film, moisture resistant skin barrier formulation recited in claim 5 wherein said film-forming solute further includes a plasticizer.

8. The adhesion-promoting, thin film, moisture resistant skin barrier formulation recited in claim 2 wherein said formulation further includes at least one optional ingredient, said at least one optional ingredient being one or more of an antimicrobial agent or bioactive agent.

9. The adhesion-promoting, thin film, moisture resistant skin barrier formulation recited in claim 8 wherein said at least one optional ingredient is benzalkonium chloride.

10. An adhesion-promoting, thin film, moisture resistant skin barrier formulation comprising
a fugitive solvent mixture consisting of t-butanol and an alkane fraction consisting of at least one alkane, wherein the t-butanol is at least 40% by weight of the fugitive solvent mixture and is less than 50% by weight of the formulation; and
a film-forming solute dissolved in the fugitive solvent mixture.

11. The adhesion-promoting, thin film, moisture resistant skin barrier formulation recited in claim 10 wherein said formulation further includes at least one of an antimicrobial agent or bioactive agent dissolved in the fugitive solvent mixture.

12. The adhesion-promoting, thin film, moisture resistant skin barrier formulation recited in claim 11 wherein said alkane fraction consists of iso-octane.

13. The adhesion-promoting, thin film, moisture resistant skin barrier formulation recited in claim 12 wherein said film-forming solute consists of butyl ester of poly-vinyl-methyl-ether/maleic acid copolymer.

14. The adhesion-promoting, thin film, moisture resistant skin barrier formulation recited in claim 13 wherein said at least one agent is benzalkonium chloride.

15. The adhesion-promoting, thin film, moisture resistant skin barrier formulation recited in claim 14 wherein the iso-octane is 55.0% by weight of said formulation, the t-butanol is 41.1% by weight of said formulation, said film-forming solute is 3.4% by weight of said formulation, and said at least one agent is 0.5% by weight of said formulation.

16. The adhesion-promoting, thin film, moisture resistant skin barrier formulation recited in claim 14 wherein the iso-octane is 44.0% by weight of said formulation, the t-butanol is 33.5% by weight of said formulation, said film-forming solute is 22.0% by weight of said formulation, and said at least one agent is 0.5% by weight of said formulation.

17. The adhesion-promoting, thin film, moisture resistant skin barrier formulation recited in claim 10 wherein said formulation has an adhesive strength greater than seven inch-pounds.

18. The adhesion-promoting, thin film, moisture resistant skin barrier formulation recited in claim 10 wherein said formulation has an evaporation time on human skin less than twenty seconds.

19. The adhesion-promoting, thin film, moisture resistant skin barrier formulation recited in claim 18 wherein said formulation has an evaporation time on human skin less than eight seconds.
